(19) United States
(12) Patent Application Publication
(10) Pub. No.: US 2025/0326783 A1
(43) Pub. Date: Oct. 23, 2025

(54) DISCOVERY OF A URIDINE RNA MODIFICATION AND A PROPOSED BIOSYNTHESIS PATHWAY

(71) Applicant: New York Institute of Technology, Old Westbury, NY (US)

(72) Inventor: Shenglong Zhang, Fort Lee, NJ (US)

(21) Appl. No.: 18/965,274

(22) Filed: Dec. 2, 2024

Related U.S. Application Data
(60) Provisional application No. 63/604,511, filed on Nov. 30, 2023.

(51) Int. Cl. C07H 19/02; A61K 31/7115; A61K 39/00; C12N 9/10; C12P 19/38; C12Q 1/6872

(52) U.S. Cl. CPC C07H 19/02; A61K 31/7115; A61K 39/00; C12N 9/1085; C12P 19/385; C12Q 1/6872; C12Y 205/01025; A61K 2039/53; C12Q 2600/118

(57) ABSTRACT

The present disclosure relates to the identification of a novel nucleoside modification, referred to herein as anhydride U, present in tRNA molecules within the cell. The present disclosure further relates to the enzymatic and chemical production of anhydride U as well as methods for detecting and regulating the presence of anhydride U within the cell.

### CROSS REFERENCE TO RELATED APPLICATIONS

This application claims the benefit of and priority to U.S. Provisional Application No. 63/604,511 filed on Nov. 30, 2023, the entire contents of which being incorporated by reference herein in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 8, 2025, is named “2637-8 US.xml” and is 20,153 bytes in size. The sequence listing contained in this .XML file is part of the specification and is hereby incorporated by reference herein in its entirety.

### TECHNICAL FIELD

The present disclosure relates to the identification of a novel nucleoside modification, referred to herein as anhydride U (also referred to herein as U′), present in tRNA molecules within the cell. The present disclosure further relates to the enzymatic and chemical production of anhydride U as well as methods for detecting and regulating the presence of anhydride U within the cell.

### BACKGROUND

It has been over sixty years since the discovery of the first RNA modification pseudouridine (Ψ) once called the fifth nucleoside.^{1,2} Since then, cutting-edge liquid chromatography mass spectrometry (LC-MS) has promoted the detection of a variety of new ribonucleotide forms. Currently three hundred and thirty-four naturally occurring nucleoside modifications have been discovered of which one hundred and eighteen are found in uridine.³

The structure of RNA depends on the sequence of nucleotides and their interactions, and in tRNA, base pairing directs the formation of a cloverleaf structure.⁴ This cloverleaf structure further folds over onto itself, and portions of the D-loop and T Ψ C-loop come into contact; nucleotide modifications in this region may provide regional stability or flexibility.^{5,6}

Since their discovery, research into the role, function, evolution, structure, and synthesis of modified RNAs is ongoing, as well as their involvement in human disease, immune response, and changes during cellular stress. In order to locate, identify and quantify these RNA modifications, direct sequencing methods involving mass spectrometry are continually under development. Accordingly, the discovery of novel modified nucleosides in RNA, along with novel methods for formation of such naturally occurring nucleosides are needed.

### SUMMARY

The present disclosure is based on the discovery of a 307 Da (unit mass) anhydride U modification (used interchangeably with U′ herein) present in tRNA^{cys} at position 19. The structure of the anhydride U is depicted in (FIG. 3A). Further, it is observed that, at position 19 in tsRNA^{Cys} and tRNA^{Cys}, acp³U and anhydride U coexist. While not being bound to any particular theory, it is believed that the presence of such an anhydride U plays a role in the stabilization of the tRNA tertiary structure.

The present disclosure provides systems, methods and compositions for the production of anhydride U and anhydride U containing nucleic acids. In one embodiment, the disclosure proves systems and methods for enzymatically synthesizing anhydride U. In this embodiment, the system can include a substrate comprising a quantity of isolated uridine and S-adenosyl methionine to which aminocarboxypropyltransferase is added leading to the production of 3-3-amino-3-carboxypropyfluoride (acp3U) which is then converted to anhydride U. In some embodiments the anhydride nucleoside is isolated, purified or biologically pure.

In some embodiments, the disclosed methods comprise generating a modified anhydride U in a tRNA molecule. In particular embodiments, methods of the disclosure comprise incubation of a nucleic acid molecule (e.g., an RNA molecule comprising uridine, or a RNA comprising acp³U under conditions sufficient for generation of a modified anhydride U.

In another embodiment, RNA therapies are provided that can be used to treat a variety of different diseases and disorders. For example, different RNA-based strategies are provided to generate novel therapeutics, including antisense and RNAi-based mechanisms, mRNA-based approaches, and CRISPR-Cas-mediated genome editing. Accordingly, in specific embodiments, compositions are provided comprising therapeutic nucleic acid molecules, e.g., RNA molecules designed to inhibit, silence or attenuate the expression of target RNAs within a cell, e.g., antisense RNA-based therapeutics. Additionally, said therapeutic RNA molecules may include CRISPR-RNAs. Such compositions include therapeutic RNAs comprising anhydride U which can enhance their stability, efficacy, and efficiency, thereby significantly advancing gene therapy and other RNA-based therapies that utilize such therapeutic nucleic acid molecules. Any of the anhydride U containing nucleic acids, provided herein, may be used in therapeutic methods described herein.

In one embodiment, a pharmaceutical composition comprising an anhydride U containing nucleic acid molecule and a pharmaceutically acceptable carrier is provided herein. Said pharmaceutical composition may be administered to a subject wherein the expression of a target RNA is associated with a particular disease or disorder. Through administration of, for example, antisense RNA comprising anhydride U the expression of the target RNA may be down regulated as a means for treatment of the particular disease or disorder. Additionally, in connection with the use of CRISPR-RNAs, said RNAs may be administered to a subject as a for gene therapy with the goal of correction of a genetic defect associated with a particular disease or disorder.

The present disclosure further relates to compositions and methods for prevention and/or treatment of diseases or disorders found to be associated with either an increase or decrease in tRNA molecules comprising modified nucleosides. In an embodiment, the modified nucleoside is an anhydride U. In one embodiment, such treatments are designed to reduce the presence of anhydride U containing tRNA molecules. Such treatment may include the use of antisense RNAs. In another embodiment, such treatments are designed to increase the presence of anhydride U containing tRNA molecules. Such treatments may include the administration of anhydride U containing tRNAs, or nucleic acids encoding said tRNAs.

In yet another embodiment, vaccine formulations are provided comprising anhydride U containing RNA molecules. Such RNAs comprise anhydride U which can enhance their stability, efficacy, and efficiency, thereby significantly advancing their use in vaccine formulations. Said anhydride U containing RNAs can be those RNAs expressed by a pathogen and encoding one or more pathogen proteins. The present disclosure provides, through the use of such vaccines, methods of generating an immune response in a subject to a vaccine formulation of the present disclosure.

Further aspects of the disclosure are directed to methods for detection and/or quantification of anhydride U. Such methods may include, for example, determining the position of an anhydride U in an RNA and quantifying the amount of anhydride in a population of RNA molecules. In a specific embodiment a method referred to as MLC-SEQ can be used to detect and quantify anhydride U present within a sample. Additionally, a mass spectrometry (NGMS)-Seq platform may be used for the sequencing of a specific tRNAs.^{8,9}

RNA plays essential roles in not only translating nucleic acids into proteins, but also in gene regulation, environmental interactions and many human diseases. For example, ERNA modifications may play a role in modulating gene expression and deregulated expression of the RNA modifications can lead to human diseases including cancer. Accordingly, the present disclosure provides methods that may be useful for evaluating nucleic acids for clinical, diagnostic, or research purposes. Certain embodiments relate to a method for evaluating a sample comprising RNA molecules to be detected. Example RNA molecules which may be analyzed using the disclosed methods and compositions include tRNAs. The evaluation may be used for the detection or determination of a particular modified nucleoside, such as anhydride U, within the sample. In some embodiments, the methods of the disclosure can be used in the discovery of novel biomarkers, i.e., anhydride U, associated with a particular disease or condition. In some embodiments, the methods of the disclosure can be performed on a sample from a patient to provide a prognosis for a certain disease or condition in the patient. In some embodiments, the methods of the disclosure can be performed on a sample from a patient to predict the patient's response to a particular therapy.

Certain aspects of the present disclosure also concern kits containing the compositions disclosed herein or compositions to implement methods disclosed herein. In some embodiments, disclosed are kits that can be used to modify and/or detect anhydride U in a target RNA. In other embodiments, the kits may be used to treat diseases or disorders known to be associated with aberrant gene expression. In another embodiment, the kits may contain vaccine compositions that comprise anhydride U RNAs encoding pathogen expressed proteins.

### BRIEF DESCRIPTION OF THE DRAWINGS

This patent or application file contains at least one drawing executed in color. Copies of this patent or patent application publication with color drawing(s) will be provided by the Office upon request and payment of the necessary fec. Various embodiments of methods are described herein with reference to the drawings wherein:

FIG. 1A-G. Evidence of a new 307 Da nucleotide modification in mouse tRNA^{Cys}.

FIG. 1A. Summary of tRNA^{Cys} de novo sequencing results showing position of modifications and quantifications using a tRNA cloverleaf structure (SEQ ID NO. 1); FIG. 1B. Position of identified modified nucleotides in tRNA^{Cys}. FIG. 1C. Summary of tsRNA^{Cys} sequencing results showing position of modifications and quantifications. (SEQ ID NO. 2) FIG. 1D. Position of identified modified nucleotides in tsRNA^{Cys}. FIG. 1E. Retention time vs mass and intensity portion of the 5′ ladder sequencing results around position 19 in tRNA^{Cys} FIG. 1F. Retention time vs mass and intensity portion of the 3′ ladder sequencing results around position 19 in tsRNA^{Cys}. FIG. 1G. Retention time vs mass and intensity portion of the 5′ ladder sequencing results around position 19 in tsRNA^{Cys}.

FIG. 2A-F. Evidence of a 307 Da nucleotide modification in mouse and tRNA^{Leu}.

FIG. 2A. MLC-Seq de novo sequencing results for tRNA^{Gln} showing the position of each identified modification and quantification. (SEQ ID NO.3) FIG. 2B. Position of each tRNA^{Gln} modification. FIG. 2C. Part of a 5′ladder sequence showing the branch point at position 16. FIG. 2D. MLC-Seq sequencing results for tRNA^{Leu} showing all the RNA modifications within the full-length tRNA sequence with their quantifications. (SEQ ID NO.4) FIG. 2E. Position of each observed modification in tRNA^{Leu}. FIG. 2F. Part of tRNA^{Leu} 5′ ladder sequence showing the U′ modification starts a new branch (triangles) with a mass of 307 Da from the main ladder sequence (circles) containing the D modification.

FIG. 3A-C. Potential pathway for the formation of anhydride U and a mechanism for converting acp³U to anhydride U.

FIG. 3A. Comparison of anhydride uridine and 5,6-dihydrocytidine structures; each has a unit mass of 307 Da. FIG. 3B. The enzymatic pathway from uridine to acp³U and further to the proposed formation of anhydride uridine. FIG. 3C. Suggested stepwise reaction mechanism supporting the conversion of acp³U to anhydride U. QM(M062x/def2TZVP) optimized geometries in a vacuum are shown with the relative Gibbs free energies in parenthesis (in kcal/mol) for the hydrolysis of acp³U.

FIG. 4A-B. The new uridine modification confers stability in tRNA^{Cys}.

FIG. 4A. Model of yeast tRNA^{Phe} (PDB ID 1ehz.pdb) generated using ChimeraX showing the interaction of the D-loop in magenta and the T Ψ C-loop in purple. FIG. 4B. 56% U′ and 44% acp³U in tRNA^{Cys} was calculated using six 5′ ladder fragments after the branch point position; similarly, 5% U′ and 95% acp³U in tsRNA^{Cys} was calculated from five 5′ladder fragments, 38% U′ and 62% D was calculated from six 5′ladder fragments in tRNA^{Gln} after the branch point position; whereas, 68% U′ and 32% U in tRNA^{Leu} was calculated from five 5′ladder fragments.

FIG. 5. Chemical approach for synthesis of anhydride U.

### DETAILED DESCRIPTION

Although the present disclosure will be described in terms of specific embodiments, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

### Definitions

The terms “transfer ribonucleic acid” or “tRNA” refer to RNA molecules with a length of typically 73 to 90 nucleotides, which mediate the translation of a nucleotide sequence in a messenger RNA into the amino acid sequence of a protein. tRNAs are able to covalently bind a specific amino acid at their 3′ CCA tail at the end of the acceptor stem, and to base-pair via a usually three-nucleotide anticodon in the anticodon loop of the anticodon arm with a usually three-nucleotide sequence (codon) in the messenger RNA. The secondary “cloverleaf” structure of tRNA comprises the acceptor stem binding the amino acid and three arms (“D arm”, “T arm” and “anticodon arm”) ending in loops (D loop, T loop (TΨC loop), anticodon loop), i.e. sections with unpaired nucleotides. The terms “D stem”, “T stem” (or “TΨC stem”) and “anticodon stem” (also “AC stem”) relate to portions of the D arm, T arm and anticodon arm, respectively, with paired nucleotides. Yeast tRNA^{Phe} has long been a model for tRNA tertiary structure, the nucleotides at position 18 and 19 in the tRNA^{Phe} D-loop form hydrogen bonds with T Ψ C-loop nucleotides at position 55 and 56, respectively; nucleotide bases at position 16 are 17 are oriented away from the L shaped structure.⁵

A nucleoside consists of a nitrogenous base covalently attached to a sugar (ribose or deoxyribose) but without the phosphate group. A nucleotide consists of a nitrogenous base, a sugar (ribose or deoxyribose) and one to three phosphate groups. A nucleotide is the basic building block of nucleic acid (RNA and DNA) molecules. The disclosure below is described for RNA but may be applied equally well to other forms of nucleic acids.

The terms “modifications” or “nucleotide modifications” used herein in relation to a RNA of the invention relates to single modified nucleotides, combinations of two or more modified nucleotides not forming base-pairs, or to one or more pairs of modified nucleotides. The terms “pair of modified nucleotides” or “modified nucleotide pair” relate to two modified nucleotides forming a base-pair within the RNA, when correctly folded. Such RNAs include, for example, mRNAs, antisense RNAs and tRNA.

The terms “isolated”, “purified”, or “biologically pure” as used herein, refer to material that is substantially or essentially free from components that normally accompany the material in its native state or when the material is produced.

As used herein, the terms “treating” and “treatment” have their ordinary and customary meanings, and include one or more of, ameliorating a symptom of a disease or disorder in a subject, blocking or ameliorating a recurrence of a symptom of the disease or disorder in a subject, decreasing in severity and/or frequency a symptom of a disease or disorder in a subject.

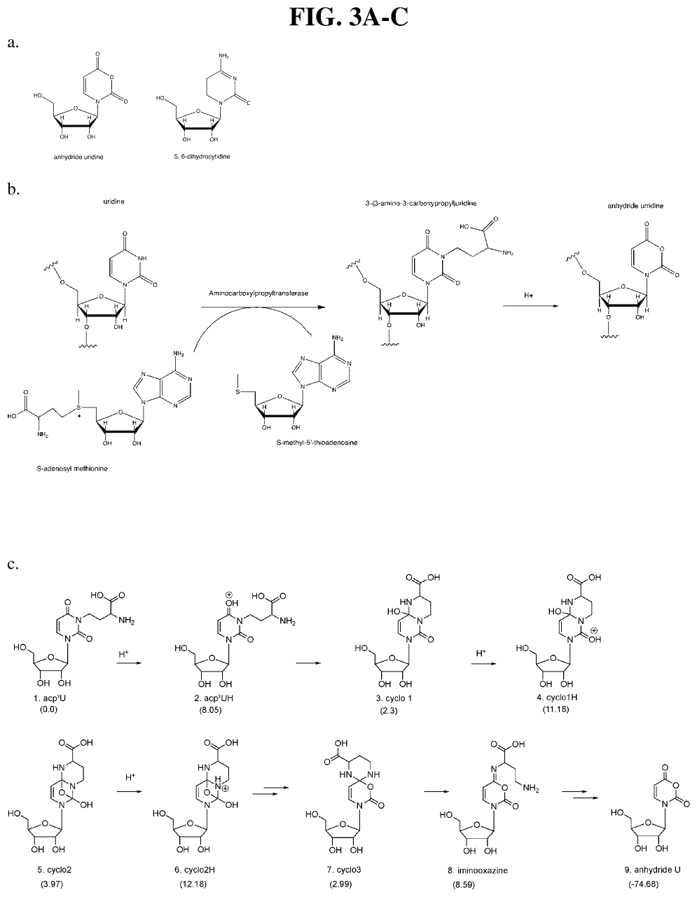

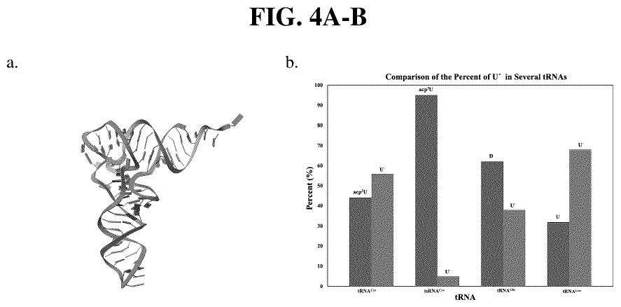

The present disclosure is based on the discovery of a 307 Da anhydride U modification present in tRNA^{cys} at position 19. The structure of the anhydride U is depicted in (FIG. 3A). Further, it is observed that, at position 19 in tsRNA^{Cys} and tRNA^{Cys}, acp³U and U′ coexist. While not being bound to any particular theory, it is believed that the presence of such a anhydride U plays a role in the stabilization of the tRNA tertiary structure.

In another embodiment, said anhydride U modification has also been detected in tRNA^{Gln} and tRNA^{Leu}. Sequencing data identified the anhydride U modification at position 16, a different D-loop position from tRNA^{Cys}, where the anhydride U modification generates a parallel branch with an earlier retention time from the main 5′ ladder sequence, indicating a second nucleotide coexists at this position. Its unit mass was confirmed to be 307 Da, a mass never reported before. This anhydride U occurs with the canonical uridine in tRNA^{Leu}; however, in tRNA_{Gln} anhydride U coexists along with the modified uridine, dihydrouridine (D). Even though the abundance and position of this 307 Da U′ modification varies in different tRNAs, the presence of anhydride U in several different tRNAs indicates that this is a commonly occurring RNA modification.

While the present disclosure is exemplified by description of anhydride U in tRNAs, it is not to be limited to such nucleic acid molecules as this unique modification may be present in other nucleic acid types including DNA, RNA nucleic acid molecules.

The present disclosure provides systems, methods and compositions for the production of anhydride U. In one embodiment, the disclosure provides systems and methods for enzymatically synthesizing anhydride U. In this embodiment, the system can include a substrate comprising a quantity of isolated uridine and S-adenosyl methionine to which aminocarboxypropyltransferase is added leading to the production of 3-3-amino-3-carboxypropyfluoride (acp3U) which is then hydrolyzed to anhydride U. In some embodiments the anhydride is isolated, purified or biologically pure.

In some embodiments, the disclosed methods comprise steps for generating a modified anhydride U in a tRNA molecule. In particular embodiments, such methods comprise incubation of a nucleic acid molecule, e.g., an RNA molecule comprising uridine, or a RNA comprising acp³U, under conditions sufficient for generation of a modified anhydride U. For example, as disclosed herein, incubation of a nucleic acid molecule at a pH between 6 and 8 may be sufficient to modify a uridine in the nucleic acid molecule to generate an anhydride U.

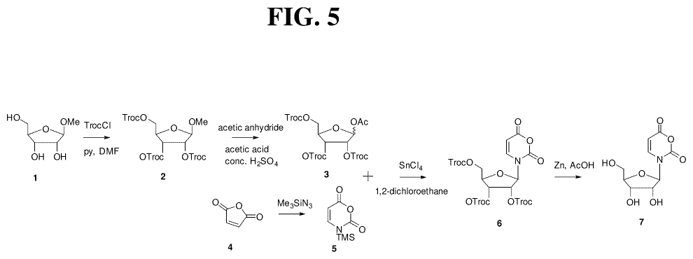

In a non-limited embodiment a chemical approach is provided for synthesis of anhydride U, a pyrimidine nucleoside analog of uridine. (See, FIG. 5) Commercial methyl β-D-ribofuranoside 1 reacts with 2,2,2-trichloroethoxycarbonylchloride in DMF and anhydrous pyridine to give troc protected sugar 2, with treatment of excess acetic acid and acetic anhydride in the presence of concentrated sulfuric acid affords acylated sugar 3. (Tek-Ling Chwang, et al. Journal of Medicinal Chemistry 1976, 19 (5), 643-647). Maleic anhydride 4 reacts with trimethysilyl azide in toluene affords silylated 3-oxauracil 5 through cyclization of a Curtius rearrangement intermediate which arises from ring opening product acyl azide. (Stephen S. Washburne et al., The Journal of Organic Chemistry 1972, 37 (11), 1738-1742). Tetra O-acylated ribofuranose 3 and N-trimethylsilyl uracil anhydride 5 react in the presence of stannic chloride and 1,2-dichloroethane to produce acylated uridine analog 6. Removal of the troc protecting group with zinc dust in acetic acid gives the final product anhydride uridine 7. (Tek-Ling Chwang, et al. Journal of Medicinal Chemistry 1976, 19 (5).

A favorable stepwise reaction mechanism was identified to support the conversion of acp3U to anhydride U. (sec, FIG. 3C). Under mild acidic conditions (pH 6), an intramolecular nucleophilic addition initiates the cyclization of a six-membered ring. This occurs when the amino group of the acp3 U (compound 1) reacts with the carbonyl carbon at position 4, leading to the formation of intermediate compound 3. Subsequently, the hydroxyl group in compound 3 attacks the carbonyl carbon at position 2 via another intramolecular nucleophilic addition at the same acidic conditions, resulting in the formation of intermediate compound 5, which contains a strained four-membered ring due to its distorted bond angles. The opening of this strained ring in compound 6 generates intermediate compound 7.Compound 7 acquires a positive charge via another ring opening, leading to a positively charged imine 8. Hydrolysis of compound 8 ultimately produces anhydride U.

Additionally, this process can occur in RNA containing acp3U under acidic conditions or with the assistance of enzymes capable of providing a proton, such as those containing an imidazole residue.

In an embodiment. RNA based therapies are provided for treatment of a variety of different diseases and disorders. For example, different RNA-based strategies are available to generate novel therapeutics, including antisense and RNAi-based mechanisms, mRNA-based approaches, and CRISPR-Cas-mediated genome editing. Additionally, mRNA vaccines have been successfully developed to combat pathogenic infections.

Accordingly, in specific embodiments, compositions are provided comprising therapeutic nucleic acid molecules, e.g., RNA molecules designed to inhibit, silence or attenuate the expression of target RNAs within a cell, e.g., antisense RNA-based therapeutics wherein the RNA comprises one or more anhydride U. Additionally, said therapeutic RNA molecules may include CRISPR-RNAs wherein the RNA comprises one or more anhydride U. The inclusion of the anhydride U can be employed to enhance the stability, efficacy, and efficiency of the RNAs, thereby significantly advancing gene therapy and other RNA-based therapies that utilize such therapeutic nucleic acid molecules.

Methods for design and expression of such nucleic acids, e.g., antisense, miRNA, siRNA and shRNA, CRISPR-RNA are well known to those of skill in the art. For example, routine methods can be used to construct expression vectors containing the coding sequence of the therapeutic RNA with appropriate transcriptional and translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). Alternatively, the therapeutic RNA molecules may chemically synthesized to include anhydride U using methods known to those skilled in the art.

Any of the anhydride U containing nucleic acids, provided herein may be used in therapeutic methods described. In one embodiment, a pharmaceutical composition comprising an anhydride U containing nucleic acid molecule and a pharmaceutically acceptable carrier is provided herein. For use in the therapeutic methods described herein, said nucleic acid molecules, of certain embodiments are formulated, dosed, and administered in a fashion consistent with good medical practice. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

Factors for consideration in this context include the particular disorder being treated, the particular subject being treated, the clinical condition of the subject, the cause of the disease or condition, the site of delivery of the agent, the method of administration, the scheduling of administration, and other factors known to medical practitioners or those of skill in the art. A “subject” or “individual” to be treated according to any of the provided embodiments is a mammal, preferably a human.

Said anhydride U containing therapeutic RNAs include, for example, tsRNA, miRNA, micro RNA, siRNA, sgRNA and CRISPR-RNA. Such therapeutic RNAs may be administered to “inhibit,” “silencing,” and/or “attenuate” target gene expression thereby leading to a measurable reduction in expression of a target RNA (or the corresponding polypeptide or protein) as compared with the expression of the target mRNA (or the corresponding polypeptide or protein) in the absence of an interfering RNA molecule of the present disclosure. Said therapeutic RNAs may be administered to subjects where the expression of a target gene is found to be associated with a particular disease or disorder. The reduction in expression of the target mRNA (or the corresponding polypeptide or protein) is commonly referred to as “knock-down” and is reported relative to levels present following administration or expression of a non-targeting control RNA. Said therapeutic RNAs include CRISPR-RNAs that may be used in gene therapy applications for editing of a subject's genome.

The term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. The term “antisense strand” is used in reference to a nucleic acid strand that is complementary to the “sense” strand.

In a non-limiting embodiment, the terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways, wherein the compounds interfere with gene expression.

In another non-limiting embodiment, the term “shRNA” refers to any small hairpin RNA or short hairpin RNA. Although it is not necessary to understand the mechanism of action, it is believed that any sequence of RNA that makes a tight hairpin turn can be used to silence gene expression via RNA interference. Typically, shRNA uses a vector introduced into a cell genome and is constitutively expressed by a compatible promoter. The shRNA hairpin structure may also be cleaved into siRNA, which may then become bound to the RNA-induced silencing complex (RISC). This complex binds to and cleaves mRNAs which match the siRNA that is bound to it.

In yet another non-limiting embodiment, the term “microRNA” or “miRNA”, refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

The present disclosure relates to compositions that comprise nucleic acid molecules designed to target mRNA and inhibit, silence or attenuate the expression of that RNA and methods for preparing them. In such instances, the nucleic acid molecules contain a region of nucleotide sequence that can direct the destruction and/or translational inhibition of the targeted transcripts. Methods for design and expression of such nucleic acids, e.g., antisense, miRNA, siRNA and shRNA, are well known to those of skill in the art. In such instances, the nucleic acid molecules contain (i) a region of nucleotide sequence that can direct the destruction and/or translational inhibition of the targeted RNA; and (ii) at least one Anhydride U.

The present disclosure relates to compositions and methods for prevention and/or treatment of diseases or disorders found to be associated with either an increase or decrease in tRNA molecules comprising modified nucleosides. In an embodiment, the modified nucleoside is an anhydride U. In one embodiment, such treatments are designed to reduce the presence of anhydride U containing tRNA molecules. In another embodiment, such treatments are designed to increase the presence of anhydride U containing tRNA molecules.

In a specific embodiment, compositions are provided comprising nucleic acid molecules designed to target a particular tRNA comprising an anhydride U and inhibit, silence or attenuate the expression of that tRNA. Such compositions may be used in methods for prevention or treatment of diseases or disorders found to be associated with aberrant levels of tRNAs comprising an anhydride U.

In another embodiment, compositions are provided comprising nucleic acid molecules designed to target a mRNA encoding the aminocarboxypropyltransferase that functions in the production of 3-3-amino-3-carboxypropyfluoride (acp3U) which is then hydrolyzed to anhydride U.

The present disclosure relates to compositions that comprise nucleic acid molecules designed to target tRNAs and inhibit, silence or attenuate the expression of that tRNA and methods for preparing them. In such instances, the nucleic acid molecules contain a region of nucleotide sequence that can direct the destruction and/or translational inhibition of the targeted transcript.

The present disclosure relates to compositions that comprise nucleic acid molecules designed to target mRNA encoding the aminocarboxypropyltransferase and inhibit, silence or attenuated the expression of that RNA and methods for preparing them. In such instances, the nucleic acid molecules contain a region of nucleotide sequence that can direct the destruction and/or translational inhibition of the targeted aminocarboxypropyltransferase transcript.

In a specific embodiment, compositions are provided comprising nucleic acid molecules designed for recombinant expression of a target a particular tRNA comprising an anhydride U to increase the expression of that tRNA. Alternatively, the compositions may contain recombinant nucleic acids designed to increase the expression of minocarboxypropyltransferase in a cell. Such compositions may be used in methods for prevention or treatment of diseases or disorders found to be associated with aberrant levels of tRNAs comprising an anhydride U.

RNA vaccines, or mRNA vaccines are a type of vaccine that uses a copy of an RNA associated with a pathogen to trigger an immune response in a vaccinated subject. Accordingly, in yet another embodiment, vaccine formulations comprising anhydride U containing RNA molecules are provided. Such RNAs are designed to include anhydride U which can enhance their stability, efficacy, and efficiency, thereby significantly advancing their use in vaccine formulations. Said anhydride U containing RNAs can be those RNAs expressed by a pathogen and encoding one or more pathogen proteins. Said pathogens include, for example, bacterial, viral, pathogenic, and yeast pathogens. The RNA vaccines comprising at least one anhydride U provides methods of using the vaccines in the treatment, prevention and prophylaxis of diseases associated with pathogen infection in a subject.

The vaccine formulations of the present disclosure comprise a full length and/or a portion of an anhydride U containing RNA encoded by a pathogen and a pharmaceutically acceptable carrier or diluent. The present disclosure provides through the use of such vaccines, methods of generating an immune response in a subject to a vaccine formulation of the present disclosure. In one embodiment, the present disclosure is directed to methods of generating an immune response in a subject, comprising administering an immunologically effective amount of a vaccine formulation of the present disclosure to a subject, thereby generating an immune response against anhydride U containing RNA in a subject. In the methods of generating an immune response of the present disclosure, the immune response is preferably a protective immune response against anhydride U containing RNA.

The present disclosure is directed to methods for providing prophylaxis of a pathogen infection in a subject using the vaccine formulations of the present disclosure. In one embodiment, the present disclosure is directed to methods for providing prophylaxis of a pathogen infection in a subject, comprising administering a therapeutically effective amount of a vaccine formulation of the present disclosure to a subject in danger of a pathogen infection, thereby providing prophylaxis of a pathogen infection in a subject.

The present disclosure is also directed to methods of treating a pathogen infection in a subject using the vaccine formulations of the present disclosure. In one embodiment, the present disclosure is directed to methods of treating a pathogen infection in a subject, comprising administering a therapeutically effective amount of a vaccine formulation of the present disclosure to a subject having a pathogen infection, thereby treating a pathogen infection in a subject.

Further aspects of the disclosure are directed to methods for detection and/or quantification of anhydride U. Such methods may include, for example, determining the position of an anhydride U in an RNA and quantifying the amount of anhydride in a population of RNA molecules. In a specific embodiment a method referred to as MLC-SEQ can be used to detect and quantify anhydride U present within a sample. (See, US2021/0217494, US2021/0198734 and US2022/0220552 each of which are incorporated herein in their entirety) In such a method, controlled acid hydrolysis of tRNA at a low pH cleaves RNA phosphodiester bonds forming two types of fragments (ladder sequences) with unique masses: 5′-ladder sequences have a 3′-phosphate group and 3′-ladder sequences have OH-groups at both ends.⁷ During LC-MS, fragments with a lower mass are separated from larger ones because they tend to elute at earlier times so during LC-MS, the ladder sequences with their associated intensities are separated by mass as well as by retention time. A plot of the deconvoluted masses (ladder sequences) and retention times (t_{R}) form a signature sigmoidal curve. Ladder sequence mass differences in the sigmoidal curve are used to identify each nucleotide by its mass in the sequence; thus, the position and identity as well as the quantity of each nucleotide in the sequence can be determined.

Additionally, a mass spectrometry (NGMS)-Seq platform may be used for the sequencing of a specific tRNAs.^{8,9} Like their previous MS-based sequencing work, mass spectrometry ladder complementation sequencing (MLC-Seq), relies on a set of ladders. These contain a series of fragments differing by one nucleotide; differences in fragment masses are used to identify the nucleotides while the ladder shows their order. Additionally, ladder branching in a 2D mass-t_{R} curve is indicative of a partial single nucleotide substitution, which can be used to provide unbiased, precise, and quantitative mapping of RNA modifications.

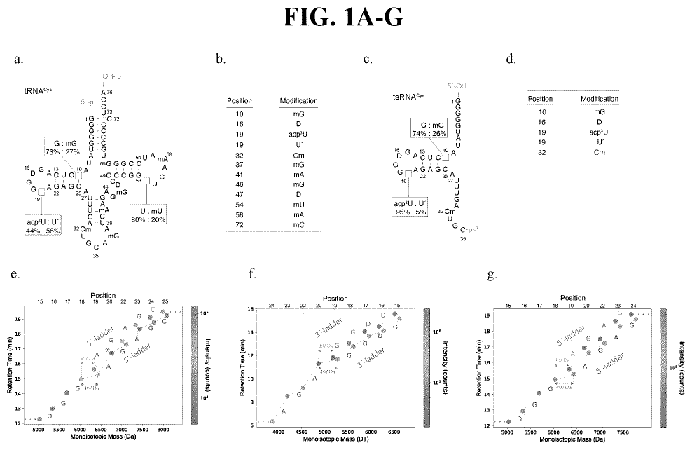

For example, the 5′ ladder tRNA^{Cys} sequencing results illustrate the parallel ladder branching effect a modification produces (FIG. 1E); a new RNA modification with a unit mass of 307 Da was detected during de novo sequencing in both of the tRNA^{Cys} and tsRNA^{Cys} mouse liver extract samples in the D-loop at position 19 (FIG. 1). The nucleoside 3-(3-carboxypropyl) uridine (acp³U) was found to coexist with U′ at this position.

It is worth noting that the 5′ ladders can also be distinguished from the 3′ ladders, because they generally have higher retention times. Unlike tRNA^{Cys} 3′ ladder fragments which contain hydroxyl groups at both ends, the tsRNA^{Cys} contain 3′ phosphates. Because their 5′ and 3′ ladder fragments are not the same, their sigmoidal curves are also discernible; the presence of a coexisting nucleotide leads to the formation of a parallel sigmoidal branch. The 5′ and 3′ ladder tsRNA sequencing results further support the existence of this observed new RNA modification (FIG. 1F and 1G) identified by a unique mass difference in each ladder sequence, coexisting with acp³U after the branch point of a parallel sigmoidal curve. The tRNA cloverleaf structure is illustrated using the current conventional numbering system.

RNA plays essential roles in not only translating nucleic acids into proteins, but also in gene regulation, environmental interactions and many human diseases. For example. RNA modifications may play a role in modulating gene expression and deregulated expression of the RNA modifications can lead to human diseases including cancer. Accordingly, the present disclosure provides for the application of anhydride U as a biomarker or molecular target, along with associated proteins, thereby providing methods for precision medicine and enabling personalized approaches for disease diagnosis and treatment. In a specific embodiment, the present disclosure provides methods that may be useful for evaluating nucleic acids for clinical, diagnostic, or research purposes. Certain embodiments relate to a method for evaluating a sample comprising RNA molecules to be detected. Example RNA molecules which may be analyzed using the disclosed methods and compositions include tRNAs. The evaluation may be used for the detection or determination of a particular modified nucleosides, such as anhydride U, within the sample.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. In some embodiments, the sample comprises cell-free RNA. Cell-free nucleic acid may be isolated, extracted, or otherwise purified from a biological sample for further analysis or processing using methods well known in the art.

In some embodiments, the methods of the disclosure can be used in the discovery of novel biomarkers, i.e., anhydride U, for a disease or condition. In some embodiments, the methods of the disclosure can be performed on a sample from a patient to provide a prognosis for a certain disease or condition in the patient. In some embodiments, the methods of the disclosure can be performed on a sample from a patient to predict the patient's response to a particular therapy.

In such diagnostic methods, a decrease in detection of a modified nucleoside, within a tRNA, may signal the presence of a disease or disorder know to correlate to such a decrease. In another embodiment, an increase in detection of a modified nucleoside, within a tRNA, may signal the presence of a disease or disorder known to correlate to such a decrease.

In such prognostic methods, a decrease in detection of a modified nucleoside, within a tRNA, may signal prognosis for recovery from a disease or disorder known to correlate to such a decrease. In another embodiment, an increase in detection of a modified nucleoside, within a tRNA, may signal the prognosis for recovery from a disease or disorder known to correlate to such a decrease.

In such prognostic methods, a decrease in detection of a modified nucleoside, within a tRNA, may signal prognosis for recovery from a disease or disorder known to correlate to such a decrease. In another embodiment, an increase in detection of a modified nucleoside, within a tRNA, may signal the prognosis for recovery from a disease or disorder known to correlate to such a decrease.

In yet another embodiment, detection and quantification of modified nucleosides, such as anhydride U, may be used to monitor a patient's response to a drug treatment administered to treat a disease or disorder. In such a method, a decrease in detection of a modified nucleoside, within a tRNA, may signal a positive response to a drug treatment for a disease or disorder known to correlate to such a decrease. In such a method, an increase in detection of a modified nucleoside, within a tRNA, may signal a positive response to a drug treatment for a disease or disorder known to correlate to such an increase.

In embodiments of the present disclosure, methods are provided for screening and identification of compounds that regulate the incorporation of anhydride U into nucleic acid molecules. Such compounds may be inhibitors of incorporation of anhydride U into nucleic acids. Alternatively, such compounds may be enhancers of incorporation of anhydride U into nucleic acids. The methods may be used to screen for compounds that inhibit or activate the enzymes involved in the synthesis, removal, or binding of anhydride U within a nucleic acid. Such enzymes can serve as valuable drug targets.

In a specific example, the screening method comprises the steps of contacting a test sample of cells, or a test animal, with a test compound and comparing the level of anhydride U observed in the test sample to a control sample. Methods for detecting the level of anhydride U in a sample are described above. A “compound” or “test compound” is any substance or any combination of substances that is useful for achieving an end or result. Any compound that has the potential to modulate through inhibition or enhancement of incorporation of anhydride U in a nucleic acid can be tested using the methods of this disclosure. Exemplary test compounds include, but are not limited to, peptides, such as soluble peptides, including but not limited to members of random peptide libraries, antibodies and antibody fragments and small organic or inorganic molecules. Appropriate test compounds can be contained in libraries, for example, synthetic or natural compounds in a combinatorial library.

Certain aspects of the present disclosure also concern kits containing compositions of the disclosure or compositions to implement methods disclosed herein. In some embodiments, disclosed are kits that can be used to modify and/or detect anhydride U in a target RNA. In other embodiments, the kits may be used to treat diseases or disorders known to be associated with aberrant expression of RNAs associated with a particular disease or disorder including, for example, tRNAs containing anhydride U. In yet another embodiment, the kit may contain vaccines wherein said vaccines are RNA based and comprise anhydride RNA.

The kit may comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, and/or preventing diseases associated with aberrant expression of RNAs comprising anhydride U. The kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In another embodiment, kits are provided for detecting the presence in a sample of tRNAs containing anhydride U. Components that are useful for processing a sample for analysis, i.e., purifying, amplifying, or sequencing the tRNA, may be included in the kit.

### EXAMPLE 1

### Materials and Methods

Materials. All chemicals were purchased from Sigma Aldrich, except 1,1,1,3,3,3-hexafluoro-2-propanol (HFIP) and N, N-diisopropylethylamine (DIPEA) which were from Thermo Fisher Scientific, and formic acid which was from Merk. The RNA samples were kindly provided for by Dr. Qi Chen.

Acid Hydrolysis. Samples were first aliquoted into individual tubes and degraded as previously described. Formic acid hydrolysis was performed on selected tubes at 40° C. using different time points, and then placed on dry ice. After being dried in a speedvac at 4° C.; all samples were then stored in a freezer.

Reversed phase liquid chromatography. A DNAPac RP column (4 um, 2.1×100 mm) was purchased from Thermo Fisher (Waltham, MA, USA) and installed in a Vanqiush Horizon UHPLC system (Thermo Fisher, Waltham, MA, USA). Mobile phase A was prepared with 15 mM TEA and 50 mM HFIP buffers in aqueous solution. Mobile phase B was prepared at the ratio of 50:50 water/methanol with buffer 7.5 mM TEA and 25 mM HFIP. During LC analysis, the column temperature was maintained at 60° C. and the flow rate was set at 0.3 mL/min. The composition of mobile-phase B was started at 5% and increased to 10% in 5 min. Then it was increased to 25% in 20 min and again raised to 50% in another 15 min. Five microliters of RNA solutions were injected by the autosampler without dilution.

Mass spectrometry. Before LC-MS, hydrolyzed samples were resolubilized in nuclease free water and pooled. A Thermo Fisher Scientific Orbitrap Exploris 240 coupled to a Vanguish Horizon UHPLC was used to process all of the samples. Intact samples were separated over a DNAP column (2.1 mm×50 mm) using a 9 minute gradient from 20% A (2% HFIP/0.1% DIPEA) to 80% B (methanol/0.075% HFIP/0.0375% DIPEA) at a flow rate of 200 uL a minute. The hydrolyzed samples were run using a longer 39 minute gradient from 15% A to 25% B. The temperature of the column was 70° C. The mass spectrometer was set to detect masses with a mass to charge (m/z) of 580 to 3200 in the negative ion mode.

Data Analysis. Thermo Fisher Scientific BioPharma software was used to deconvolute the mass spectrometry data, and MLC-Seq was used to generate sequence information. MLC-Seq is available at https://github.com/rnamodifications/MLC-Seq.

### Results

Discovery of U′ in tRNA^{Cys} with unit mass of 307 Da.

Transfer RNAs (tRNA) are small modified non-coding RNAs from which tRNA-small RNAs (tsRNA) are derived. Controlled acid hydrolysis of tRNA at a low pH cleaves RNA phosphodiester bonds forming two types of fragments (ladder sequences) with unique masses: 5′-ladder sequences have a 3′-phosphate group and 3′-ladder sequences have OH-groups at both ends.⁷ During LC-MS, fragments with a lower mass are separated from larger ones because they tend to elute at earlier times so during LC-MS, the ladder sequences with their associated intensities are separated by mass as well as by retention time. A plot of the deconvoluted masses (ladder sequences) and retention times (t_{R}) form a signature sigmoidal curve. Ladder sequence mass differences in the sigmoidal curve are used to identify each nucleotide by its mass in the sequence; thus, the position and identity as well as the quantity of each nucleotide in the sequence can be determined.

Preliminary work resulted in the creation of a next generation mass spectrometry (NGMS)-Seq platform for the sequencing of a few specific tRNAs.^{8,9} Like previous MS-based sequencing work, mass spectrometry ladder complementation sequencing (MLC-Seq), relies on a set of ladders. These contain a series of fragments differing by one nucleotide; differences in fragment masses are used to identify the nucleotides while the ladder shows their order. Additionally, it was described how ladder branching in a 2D mass-t_{R} curve is indicative of a partial single nucleotide substitution, which can be used to provide unbiased, precise, and quantitative mapping of RNA modifications.

MLC-Seq was used to analyze tRNA mass spectrometry data in order to obtain a complete sequence coverage. MLC-Seq generation of single nucleotide sequencing information along sigmoidal curves facilitated the identification and quantification of various modifications. When a site specific partial modification is encountered, both a shift in mass and retention time occur for all of the ladder fragments containing the modification.

The 5′ ladder tRNA^{Cys} sequencing results illustrate the parallel ladder branching effect a modification produces (FIG. 1E); a new RNA modification with a unit mass of 307 Da was detected during de novo sequencing in both of the tRNA^{Cys} and tsRNA^{Cys} mouse liver extract samples in the D-loop at position 19 (FIG. 1). The nucleoside 3-(3-carboxypropyl) uridine (acp³U) coexists with U′ at this position, and it is possible that this new RNA modification is derived from uridine.

It is worth noting that the 5′ ladders can also be distinguished from the 3′ ladders, because they generally have higher retention times. Unlike tRNA^{Cys} 3′ ladder fragments which contain hydroxyl groups at both ends, the tsRNA^{Cys} contain 3′ phosphates. Because their 5′ and 3′ ladder fragments are not the same, their sigmoidal curves are also discernible; the presence of a coexisting nucleotide leads to the formation of a parallel sigmoidal branch. The 5′ and 3′ ladder tsRNA sequencing results further support the existence of this new RNA modification (FIG. 1F-G) identified by a unique mass difference in each ladder sequence, coexisting with acp³U after the branch point of a parallel sigmoidal curve. The tRNA cloverleaf structure is illustrated using the current conventional numbering system.

U′ is a common RNA modification found in other tRNAs.

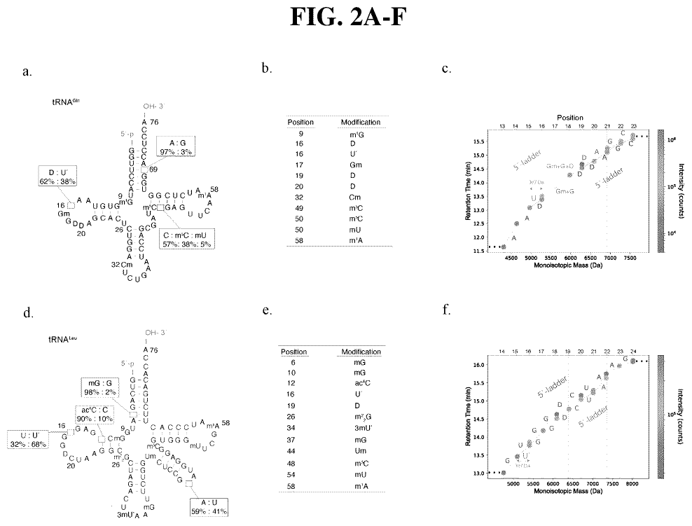

The 307 Da U′ modification is also present in mouse liver tRNA^{Gln} and tRNA^{Leu} (FIG. 2). Sequencing data identified the U′ modification at position 16, a different D-loop position from tRNA^{Cys}, where the U′ modification generates a parallel branch with an earlier retention time from the main 5′ ladder sequence (FIG. 2C; FIG. 2F), indicating a second nucleotide coexists at this position. Its unit mass was confirmed to be 307 Da, a mass never reported before. This U′ occurs with the canonical uridine in tRNA^{Leu}; however, in tRNA^{Gln} U′ coexists along with the modified uridine, dihydrouridine (D). These findings suggest U′ is somehow related to uridine.

Even though the abundance and position of this 307 Da U′ modification varies in different tRNAs, the presence of U′ in several different tRNAs indicates that this is a commonly occurring RNA modification. The role this U′ modification plays in tRNA^{Gln} and tRNA^{Leu} will most likely be different from tRNA^{Cys} since it is located at a different position in the loop and will not influence D-loop and T Ψ C-loop interactions in the same way. Yeast tRNA^{Phe} has long been a model for tRNA tertiary structure, the nucleotides at position 18 and 19 in the tRNA^{Phe} D-loop form hydrogen bonds with T Ψ C-loop nucleotides at position 55 and 56, respectively; nucleotide bases at position 16 are 17 are oriented away from the L shaped structure.⁵

Evidence to support the biosynthesis of U′ tRNA modification.

This new U′ modification has only been observed coexisting with uridine or some other type of uridine modification in the D-loop. Although a unit mass of 307 Da alone cannot determine the chemical structure of this new modification, given the two most likely chemical structures with the same mass—anhydride U and 5, 6-dihydrocytidine, we decidedly prefer anhydride U (FIG. 3A).

At position 19 in tsRNA^{Cys} and tRNA^{Cys}, acp³U and U′ coexist even after controlled acid hydrolysis and further processing indicating this modification is most likely not a product of sample handling. This indicates that 1) both modifications are naturally occurring modifications, and 2) acp³U and U′ are most likely related. In contrast, there is no feasible pathway to generate 5, 6-dihydrocytidine from U, whereas there is a stepwise reaction mechanism to support the conversion of acp³U to anhydride U, and this could occur with the aid of an enzyme (FIG. 3B-3C.). A structure is proposed for this newly discovered nucleotide U′ (FIG. 3) based on the unique mass and possible synthesis pathway.

The enzyme aminocarboxylpropyltransferase is responsible for the conversion of U to acp³U with the addition of the cofactor S-adenosylmethionine.¹² In order to confirm an acp³U conversion to U′ experiments were conducted to make a standard anhydride U, and calculate the free energy of this conversion. Theoretically, the formation of the nucleoside anhydride U from an acp³U nucleoside has a favorable Gibbs free energy. However, since this modification occurs in a nucleotide sequence, the assumption that this is an adequate representation and the formation of anhydride U in a sequence will also be favorable has been made. To confirm this new modification the following can be used: 1) the synthetic standard anhydride U nucleoside, and 2) complete degradation of tRNA into single nucleotides and further nucleosides using enzymes (not formic acid which is typically use to generate ladders for MS sequencing) and 3) MS/MS to differentiate U from U′ using the standard anhydride U.

Anhydride U stabilizes tsRNA^{Cys} precursor tRNA^{Cys}

The tertiary structure of tRNAs is L-shaped, and in order for this to happen the D and T Ψ C-loop come together, making some of the loop bases less accessible. Many modifications are important for tRNA structure providing regions of stability and flexibility. The nucleotide modification 7-methylguanosine (m⁷G) at position 46 is known to provide a slightly more stabilized tRNA structure by base pairing with cysteine (C) at position 13 and glutamine (G) at position 22; whereas, a dihydrouridine (D) present in the D loop allows for some flexibility because the C2′endoribose conformation is favored.^{13,14} These interactions are also present in mouse tRNA^{Cys}. Some modifications also directly affect protein synthesis; for instance; the loss of 1-methylguanosine (m¹G) at position 37 can cause frameshifts which impacts translation efficiency.¹⁵ Other modification such as N¹-methyladenosine (m¹A) are reversible, enabling tRNA to be functionally regulated; whereas 5-methylcytosine (m⁵C) can inhibit stress induced tRNA cleavage.^{16,17}

After analyzing the sequencing data, over a ten-fold higher stoichiometry of the new U′ modification in tRNA^{Cys} (56%) than in the related tsRNA^{Cys} (5%) was found. An incorporation of U′ at position 19 in the D-loop could possibly protect tRNA^{Cys} from a tsRNA^{Cys} conversion by altering the D-loop and T Ψ C-loop interactions and subsequent structure of RNA. Any change in tRNA^{Cys} structural stability or flexibility may prevent certain types of associations, such as those with enzymes.

The major form coexisting with the canonical uridine in tRNA^{Leu} is U′ (68%); however, in tRNA^{Gln} U′ is a minor component (38%) occurring with dihydrouridine (D). Since these nucleotides are both at a different position, 16 in the L-shaped structure, they may participate in other types of interactions; further research into the role and function of this new U′ modification is still needed.

The documents listed below and referenced herein are incorporated herein by reference in their entireties, except for any statements contradictory to the express disclosure herein, subject matter disclaimers or disavowals, and except to the extent that the incorporated material is inconsistent with the express disclosure herein, in which case the language in this disclosure controls. Incorporation by reference of the following shall not be considered an admission by the applicant that the incorporated materials are prior art to the present disclosure, nor shall any document be considered material to patentability of the present disclosure.

### Reference

1. Cohn, W., and Volkin, E. Nucleoside-5′-Phosphates from Ribonucleic Acid. Nature 167, 483-484 (1951).
2. Davis, F. F., & Allen, F. W. Ribonucleic acids from yeast which contain a fifth nucleotide. The Journal of biological chemistry, 227(2), 907-915 (1957).
3. Boccaletto, P., Stefaniak, F., Ray, A., Cappannini, A., Mukherjee, S., Purta, E., Kurkowska, M., Shirvanizadeh, N., Destefanis, E., Groza, P., Avşar, G., Romitelli, A., Pir, P., Dassi, E., Conticello, S. G., Aguilo, F., & Bujnicki, J. M. MODOMICS: a database of RNA modification pathways. Nucleic acids research, 50(D1), D231-D235 (2022).
4. Holley R. W. The nucleotide sequence of a nucleic acid. Scientific American, 214(2), 30-39 (1966).
5. Kim, S. H., Sussman, J. L., Suddath, F. L., Quigley, G. J., McPherson, A., Wang, A. H., Seeman, N. C., & Rich, A. The general structure of transfer RNA molecules. Proceedings of the National Academy of Sciences of the United States of America, 71(12), 4970-4974 (1974).
6. Shi, H., & Moore, P. The crystal structure of yeast phenylalanine tRNA at 1.93 Å resolution: A classic structure revisited. RNA, 6(8), 1091-1105 (2000).
7. Bahr, U., Aygün, H., & Karas, M. Sequencing of single and double stranded RNA oligonucleotides by acid hydrolysis and spectrometry. Analytical chemistry, 81(8), 3173-3179 (2009).
8. Zhang N., Shi S., Jia T. Z., Ziegler A., Yoo, B., Yuan X., Li W., Zhang S. A general LC-MS-based RNA sequencing method for direct analysis of multiple-base modifications in RNA mixtures. Nucleic Acids Res., 47, c125 (2019).
9. Zhang N., Shi S., Wang X., Ni W., Yuan X., Duan J., Jia T. Z., Yoo B., Ziegler A., Russo J., Li, W., and Zhang, S. Direct sequencing of tRNA by 2D-HELS-AA MS Seq reveals its different isoforms and dynamic base modifications. ACS Chem. Biol., 15, 1464-1472 (2020).
10. Choi, E. J., Wu, W., Zhang, K., Yuan, X., Deng, J., Ismail, D., Buck, D. L., Thomason, K. S., Garofalo, R.P., Zhang, S., and Bao, X. Parent tRNA Modification Status Determines the Induction of Functional tRNA-Derived RNA by Respiratory Syncytial Virus Infection. Viruses, 15(1), 57 (2023).
11. Zhang, S., Yuan, X., Su, Y., Zhang, X., Turkel, S., Shi, S., Wang, X., Choi, E., Wu, W., Liu, H., Viner, R.I., Russo, J. J., Li, W., Bao, X., & Chen, Q. MLC-Seq: de novo Sequencing of Full-Length tRNAs and Quantitative Mapping of Multiple RNA Modifications. Preprint (2021).
12. Nishimura, S., Taya, Y., Kuchino, Y., Ohashi, Z. Enzymatic synthesis of 3-(3-amino-3-carboxypropyl)uridine in Escherichia coli phenylalanine transfer RNA: transfer of the 3-amino-acid-3-carboxypropyl group from S-adenosylmethionine. Biochem. Biophys. Res. Commun., 57, 702-708 (1974).
13. Tomikawa C. 7-Methylguanosine Modifications in Transfer RNA (tRNA). International journal of molecular sciences, 19(12), 4080 (2018).
14. Dalluge J. J., Hashizume T., Sopchik A. E., McCloskey J. A., Davis D. R. Conformational flexibility in RNA: the role of dihydrouridine. Nucleic Acids Research. 24(6), 1073-1079 (1996).
15. Björk, G. R., Wikström, P. M., & Byström, A. S. Prevention of translational frameshifting by the modified nucleoside 1-methylguanosine. Science, 244(4907), 986-989 (1989).
16. Liu, F., Clark, W., Luo, G., Wang, X., Fu, Y., Wei, J., Wang, X., Hao, Z., Dai, Q., Zheng, G., Ma, H., Han, D., Evans, M., Klungland, A., Pan, T., & He, C. ALKBH1-Mediated tRNA Demethylation Regulates Translation. Cell, 167(3), 816-828.e16 (2016).
17. Schaefer, M., Pollex, T., Hanna, K., Tuorto, F., Meusburger, M., Helm, M., & Lyko, F. RNA methylation by Dnmt2 protects transfer RNAs against stress-induced cleavage. Genes & development, 24(15), 1590-1595 (2010).
18. Pettersen, E. F., Goddard, T. D., Huang, C. C., Meng, E. C., Couch, G. S., Croll, T. I., Morris, J. H., Ferrin, T. E. UCSF ChimeraX: Structure visualization for researchers, educators, and developers. Protein Sci., 30(1), 70-82 (2021).
19. Su, D., Chan, C. T., Gu, C., Lim, K. S., Chionh, Y. H., McBee, M. E., Russell, B. S., Babu, I. R., Begley, T. J., & Dedon, P. C. Quantitative analysis of ribonucleoside modifications in tRNA by HPLC-coupled mass spectrometry. Nature protocols, 9(4), 828-841 (2014).

What is claimed is:

1. An anhydride U having the structure of FIG. 3A.

2. A method for detection of a tRNA comprising the anhydride U of claim 1.

3. The detection method of claim 2 wherein the tRNA is tRNA^{Cys}.

4. The method of claim 2 wherein a MLC-SEQ method is used.

5. A method for enzymatic production of the anhydride U of claim 1, comprising (i) providing. a substrate comprising a quantity of isolated uridine and S-adenosyl methionine;
(ii) contacting said substrate to aminocarboxypropyltransferase to yield acp³U and (iii) hydrolyzing said acp³U to anhydride U.

6. A method for generating the modified anhydride U of claim 1 in a tRNA molecule comprising uridine, or a RNA comprising acp³U, comprising the step of hydrolyzing said tRNA comprising uridine, or a tRNA comprising acp³U under conditions sufficient for generation of a modified anhydride U.

7. A method for diagnostic or prognosis of a disease associated with aberrant presence of one or more tRNAs comprising the anhydride U of claim 1, comprising detection and quantification of said anhydride U in a sample.

8. A method for prevention and/or treatment of diseases or disorders found to be associated with either an increase in the anhydride U of claim 1 contained in tRNA molecules comprising administration of a composition that results in a decrease in anhydride U containing tRNAs.

9. A method for prevention and/or treatment of diseases or disorders found to be associated with either decrease in the anhydride U of claim 1 contained in tRNA molecules comprising administration of a composition that results in an increase in anhydride containing tRNAs.

10. A kit for modification and/or detection of the anhydride U of claim 1 in a target RNA.

11. A therapeutic RNA molecule comprising the anhydride U of claim 1.

12. The therapeutic RNA molecule of claim 11, wherein the therapeutic RNA is an antisense RNA that targets inhibition of a target RNA the expression of which is associated with a disease or disorder.

13. The therapeutic RNA molecule of claim 11, wherein said antisense RNA is selected from the group consisting of miRNA, micro RNA, and siRNA.

14. A pharmaceutical composition comprising the therapeutic RNA molecule of claim 11 and a pharmaceutically acceptable carrier.

15. A method of treating a disease or disorder in a subject, comprising administration of the composition of claim 14.

16. A vaccine composition comprising an RNA molecule comprising the anhydride U of claim 1.

17. The vaccine composition of claim 16, wherein the RNA molecule is expressed by a pathogen.

18. The vaccine composition of claim 17, wherein the pathogen is selected from the group consisting of a bacterial, viral, parasitic and yeast pathogen.

19. A method for screening for a compound that modulates the level of the anhydride U of claim 1 in a nucleic acid comprising the steps of (i) contacting a test sample with a test compound; and (ii) comparing the level of anhydride U in the test sample to a control sample.

20. A method for treatment of a disease or disorder associated with the expression of the anhydride U of claim 1, comprising administration of a compound that modulates the biosynthetic pathway of FIG. 3B.


SEQUENCE LISTING (4 sequences)

SEQ ID NO: 1 - RNA, 75 nt, synthetic construct
  modified_base 10: mod_base = m1g
  modified_base 16: mod_base = dhu
  modified_base 19: mod_base = OTHER; note = x or anhydride U
  modified_base 31: mod_base = cm
  modified_base 36: mod_base = m1g
  modified_base 40: mod_base = m1a
  modified_base 45: mod_base = m1g
  modified_base 46: mod_base = dhu
  modified_base 53: mod_base = OTHER; note = 1-methyl uracil
  modified_base 57: mod_base = m1a
  modified_base 71: mod_base = OTHER; note = 1-methyl cytosine
  source 1..75: mol_type = other RNA
gggggtatagctcagtggtagagcatttgactgcagatcaagaggtccccggttcaaatccgggtgccccctcca

SEQ ID NO: 2 - RNA, 34 nt, synthetic construct
  modified_base 10: mod_base = m1g
  modified_base 16: mod_base = dhu
  variation 19: note = x or anhydride U (U')
  modified_base 31: mod_base = cm
  source 1..34: mol_type = other RNA
gggggtatagctcagtggtagagcatttgactgc

SEQ ID NO: 3 - RNA, 75 nt, synthetic construct
  modified_base 9: mod_base = m1g
  variation 16: note = dhu or anhydride U
  modified_base 17: mod_base = gm
  modified_base 19: mod_base = dhu
  modified_base 20: mod_base = dhu
  modified_base 32: mod_base = cm
  modified_base 48: mod_base = m5c
  variation 49: note = m5c or 1-methyl uracil
  modified_base 57: mod_base = m1a
  variation 68: note = a or g
  source 1..75: mol_type = other RNA
ggttccatggtgtaanggttagcactctggactctgaatccagcgatcngagttcaaatctcggtggnacctcca

SEQ ID NO: 4 - RNA, 87 nt, synthetic construct
  modified_base 6: mod_base = m1g
  modified_base 10: mod_base = m1g
  modified_base 12: mod_base = OTHER; note = N4-acetylcytidine
  modified_base 16: mod_base = OTHER; note = anhydride U
  modified_base 19: mod_base = dhu
  modified_base 27: mod_base = m22g
  modified_base 35: mod_base = OTHER; note = 3-methyl anhydride U
  modified_base 38: mod_base = m1g
  modified_base 45: mod_base = um
  modified_base 49: mod_base = m5c
  misc_feature 51: note = a or u
  modified_base 59: mod_base = m5c
  modified_base 65: mod_base = OTHER; note = 1-methyl uracil
  modified_base 69: mod_base = m1a
  source 1..87: mol_type = other RNA
gtcaggatggccgagtggtctaaggcgctagacttaagttctggtctccgnatggaggcgtgggttcgaatcccacttctgacacca